SEQUENCE LISTING (14 sequences)

SEQ ID NO: 1 - protein, 285 aa, Homo sapiens
  REGION 1..285: note = misc_feature - Wild-type Dnase1L3 Primary Amino Acid sequence (amino acids 21-305 of the long isoform)
MRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 2 - protein, 285 aa, synthetic construct
  REGION 1..285: note = Synthetic: Mutant Dnase1L3 of the present invention, mutation at S91C
MRICSFNVRSFGESKQEDKNAMDVIVKVIKRCDIILVMEIKDSNNRICPILMEKLNRNSRRGITYNYVISCRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 3 - protein, 285 aa, synthetic construct
  REGION 1..285: note = Synthetic: Mutant Dnase1L3 of the present invention, mutation at S112C
MRICSFNVRSFGESKQEDKNAMDVIVKVIKRCDIILVMEIKDSNNRICPILMEKLNRNSRRGITYNYVISSRLGRNTYKEQYAFLYKEKLVCVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 4 - protein, 285 aa, synthetic construct
  REGION 1..285: note = Synthetic: Mutant Dnase1L3 of the present invention, mutation at S253C
MRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNCVFDFQKAYKLTEEEALDVSDHFPVEFKLQSSRAFTNSKKSVTLRKKTKSKRS

SEQ ID NO: 5 - protein, 285 aa, synthetic construct
  REGION 1..285: note = Synthetic: Mutant Dnase1L3 of the present invention, mutation at S91C and S112C
MRICSFNVRSFGESKQEDKNAMDVIVKVIKRCDIILVMEIKDSNNRICPILMEKLNRNSRRGITYNYVISCRLGRNTYKEQYAFLYKEKLVCVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 6 - protein, 285 aa, synthetic construct
  REGION 1..285: note = Synthetic: Mutant Dnase1L3 of the present invention, mutation at S91C and S253C
MRICSFNVRSFGESKQEDKNAMDVIVKVIKRCDIILVMEIKDSNNRICPILMEKLNRNSRRGITYNYVISCRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNCVFDFQKAYKLTEEEALDVSDHFPVEFKLQSSRAFTNSKKSVTLRKKTKSKRS

SEQ ID NO: 7 - protein, 285 aa, synthetic construct
  REGION 1..285: note = Synthetic: Mutant Dnase1L3 of the present invention, mutation at S112C and S253C
MRICSFNVRSFGESKQEDKNAMDVIVKVIKRCDIILVMEIKDSNNRICPILMEKLNRNSRRGITYNYVISSRLGRNTYKEQYAFLYKEKLVCVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNCVFDFQKAYKLTEEEALDVSDHFPVEFKLQSSRAFTNSKKSVTLRKKTKSKRS

SEQ ID NO: 8 - protein, 285 aa, synthetic construct
  REGION 1..285: note = Synthetic: Mutant Dnase1L3 of the present invention, mutation at S91C, S112C, and S253C
MRICSFNVRSFGESKQEDKNAMDVIVKVIKRCDIILVMEIKDSNNRICPILMEKLNRNSRRGITYNYVISCRLGRNTYKEQYAFLYKEKLVCVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNCVFDFQKAYKLTEEEALDVSDHFPVEFKLQSSRAFTNSKKSVTLRKKTKSKRS

SEQ ID NO: 9 - DNA, 40 bp, synthetic construct
  misc_feature 1..40: note = Synthetic: S91C primer
  source 1..40: mol_type = other DNA
cgtacaactatgtgattagctgtcggcttggaagaaacac

SEQ ID NO: 10 - DNA, 40 bp, synthetic construct
  misc_feature 1..40: note = Synthetic: S91C primer
  source 1..40: mol_type = other DNA
gtgtttcttccaagccgacagctaatcacatagttgtacg

SEQ ID NO: 11 - DNA, 38 bp, synthetic construct
  misc_feature 1..38: note = Synthetic: S112C primer
  source 1..38: mol_type = other DNA
ctacaaggaaaagctggtgtgtgtgaagaggagttatc

SEQ ID NO: 12 - DNA, 38 bp, synthetic construct
  misc_feature 1..38: note = Synthetic: S112C primer
  source 1..38: mol_type = other DNA
gataactcctcttcacacacaccagcttttccttgtag

SEQ ID NO: 13 - DNA, 38 bp, synthetic construct
  misc_feature 1..38: note = Synthetic: S253C primer
  source 1..38: mol_type = other DNA
gttcccaagtcaaactgtgtttttgacttccagaaagc

SEQ ID NO: 14 - DNA, 38 bp, synthetic construct
  misc_feature 1..38: note = Synthetic: S253C primer
  source 1..38: mol_type = other DNA
gctttctggaagtcaaaaacacagtttgacttgggaac

(19) United States
(12) Patent Application Publication
(10) Pub. No.: US 2025/0032594 A1
(43) Pub. Date: Jan. 30, 2025

(54) Improvement of Dnase1L3 Serum Half-Life

(71) Applicant: Texas Tech University System, Lubbock, TX (US)

(72) Inventors: Peter Keyel, Lubbock, TX (US); Roger B. Sutton, Lubbock, TX (US); Jon McCord, Lubbock, TX (US)

(21) Appl. No.: 18/716,609

(22) PCT Filed: Dec. 14, 2022
(86) PCT No.: PCT/US2022/081533

§ 371 Date: Jun. 5, 2024

Related U.S. Application Data
(60) Provisional application No. 63/289,789, filed on Dec. 15, 2021.

(51) Int. Cl. A61K 38/46; C12N 9/22

(52) U.S. Cl. CPC A61K 38/465; C12N 9/22

(57) ABSTRACT

The present invention includes a mutant DNase 1L3 having at least about a 95% identity with a nucleic acid sequence encoding the protein of SEQ ID NO: 2-8 for a mutant DNase 1L3 comprising at least one mutation for post-translational modification or attachment of a molecule to the mutant DNase 1L3 to increase the serum half-life of the mutant DNase 1L3, nucleic acids encoding the same, host cells, and methods of making the mutant DNase 1L3.

### CROSS-REFERENCE TO RELATED APPLICATIONS

This application claims priority to U.S. Provisional Application Ser. No. 63/289,789, filed Dec. 15, 2021, the entire contents of which are incorporated herein by reference.

### TECHNICAL FIELD OF THE INVENTION

The present invention relates in general to the field of DNase I, and more particularly to an improved mutant DNaseIL3.

### STATEMENT OF FEDERALLY FUNDED RESEARCH

None.

### INCORPORATION-BY-REFERENCE OF MATERIALS FILED ON COMPACT DISC

The present application includes a Sequence Listing which has been submitted in ST.26 format via EFS-Web and is hereby incorporated by reference in its entirety. Said ST.26 copy, created on Dec. 13, 2022, is named TECH2167WO.xml and is 21 kilo bytes in size.

### BACKGROUND OF THE INVENTION

Without limiting the scope of the invention, its background is described in connection with DNase I proteins.

One such patent is U.S. Pat. No. 9,603,907, issued to Shaaltiel, et al., entitled, “Dry powder formulations of DNase I” and is said to teach DNase I formulations for pulmonary administration and, e.g., a dry powder formulation comprising, as an active ingredient, human DNase I, methods, dry powder inhalation devices and systems for the therapeutic use thereof are provided. Further, a key limitation of DNase1 is that it poorly digests DNA present as chromatin or apoptotic bodies (see Sisirak et al, Cell 2016 166:1-14, Napirei et al 2009 FEBS J 276:1059-1073, Wilber et al 2002 Mol Ther 6:35-42).

Another such patent is U.S. Pat. No. 7,407,785, issued to Lazarus, entitled “Human DNase I hyperactive variants”, which is said to teach amino acid sequence variants of human DNase I that have increased DNA-hydrolytic activity. The invention is said to include nucleic acid sequences encoding such hyperactive variants that enable the production of these variants in quantities sufficient for clinical use, and pharmaceutical compositions and therapeutic uses of hyperactive-variants of human DNase I.

Yet another such patent is U.S. Pat. No. 7,067,298, issued to Latham, et al. entitled, “Compositions and methods of using a synthetic DNase I” and is said to teach a synthetic bovine DNase I for use in molecular biology applications, including: degradation of contaminating DNA after RNA isolation; RNA clean-up prior to, or in conjunction with, RT-PCR after in vitro transcription; identification of protein binding sequences on DNA (DNase I footprinting); prevention of clumping when handling cultured cells; tissue dissociation and creation of fragmented DNA for in vitro recombination reactions.

### SUMMARY OF THE INVENTION

As embodied and broadly described herein, an aspect of the present disclosure relates to an isolated and purified nucleic acid comprising a nucleic acid encoding a mutant DNase1L3 comprising at least one mutation for post-translational attachment of polyethylene-glycol (PEG) to the mutant DNase1L3 to increase the serum half-life of the mutant DNase1L3. In one aspect, the nucleic acid further comprises a nucleic acid sequence optimized for microbial expression. In another aspect, the mutant DNase1L3 comprises the mutant DNase1L3 comprises at least one of: D38C, A126C, Y261C, N78C, S79C, R80C, K147C, or S245C mutation. In another aspect, the mutant DNase1L3 further comprises at least one of: S91C, S112C, S131C, S253C, S272C, or S279C mutation. In another aspect, the mutant DNase1L3 comprises the mutant DNase1L3 comprises at least two mutations selected from D38C, A126C, Y261C, N78C, S79C, R80C, K147C, or S245C, S91C, S112C, S131C, S253C, S272C, or S279C mutations. In another aspect, the nucleic acid further comprises a nucleic acid segment encoding a leader sequence. In another aspect, the nucleic acid further comprises a codon-optimized mutant DNase1L3 nucleic acid encoding SEQ ID NOS: 2-8, with the at least one: D38C, A126C, Y261C, N78C, S79C, R80C, K147C, or S245C mutation. In another aspect, the nucleic acid encodes a protein that comprises an about 95, 96, 97, 98, 99, or 100 percent identity or higher with a codon-optimized mutant DNase1L3 of SEQ ID NO: 2-8, with the at least one: D38C, A126C, Y261C, N78C, S79C, R80C, K147C, or S245C, S91C, S112C, S131C, S253C, S272C, or S279C mutation.

As embodied and broadly described herein, an aspect of the present disclosure relates to an expression vector comprising a nucleic acid encoding the protein of SEQ ID NO: 2-8 for a mutant DNase1L3 comprising at least one mutation for post-translational attachment of polyethylene-glycol (PEG) operably linked to a promoter recognized by a host cell transformed with the vector. In one aspect, the host cell is a bacterial or yeast cell. In another aspect, the host cell comprises F. coli or Pichia pastoris.

As embodied and broadly described herein, an aspect of the present disclosure relates to a mutant DNase1L3 having at least about a 95% identity with a nucleic acid sequence encoding a mutant DNase1L3 comprising at least one mutation for post-translational attachment of polyethylene-glycol (PEG) to the mutant DNase1L3 to increase the serum half-life of the mutant DNase1L3. In one aspect, the mutant DNase1L3 further comprises a nucleic acid sequence optimized for microbial expression. In another aspect, the mutant DNase1L3 comprises at least one of: D38C, A126C, Y261C, N78C, S79C, R80C, K147C, or S245C mutation. In another aspect, the mutant DNase1L3 further comprises at least one of: S91C, S112C, S131C, S253C, S272C, or S279C mutation. In another aspect, the mutant DNase1L3 comprises the mutant DNase1L3 comprises at least two mutations selected from D38C, A126C, Y261C, N78C, S79C, R80C, K147C, S245C, S91C, S112C, S131C, S253C, S272C, or S279C mutations. In another aspect, the nucleic acid further comprises a nucleic acid segment encoding a leader sequence. In another aspect, the nucleic acid further comprises a codon-optimized mutant DNase1L3 nucleic acid encoding SEQ ID NOS: 2-8, with at least one: D38C, A126C, Y261C, N78C, S79C, R80C, K147C, or S245C mutation. In another aspect, the nucleic acid encodes a mutant protein that comprises an about 95, 96, 97, 98, 99, or 100 percent identity or higher with a codon-optimized mutant DNase1L3 of SEQ ID NO: 2-8, with the at least one: D38C, A126C, Y261C, N78C, S79C, R80C, K147C, or S245C mutation. In another aspect, the mutant DNase1L3 is post-translationally modified with polyethylene glycol. In another aspect, the mutant DNase1L3 is post-translationally modified with a polyethylene glycol having a molecular mass from 5 kDa to 50 kDa.

As embodied and broadly described herein, an aspect of the present disclosure relates to a host cell transformed with an expression vector comprising a nucleic acid encoding an amino acid sequence of SEQ ID NO: 2-8 for a mutant DNase1L3, with the at least one: D38C, A126C, Y261C, N78C, S79C, R80C, K147C, or S245C mutation. In one aspect, the host cell comprises a bacterial or a yeast cell. In another aspect, the host cell comprises E. coli, Pichia pastoris, or host strains that allow enhanced disulfide bond formation and enhanced expression of eukaryotic proteins that contain codons rarely used in E. coli.

As embodied and broadly described herein, an aspect of the present disclosure relates to a process for making a protein with DNase activity comprising the steps of: transforming a host cell with an isolated nucleic acid comprising a nucleotide sequence encoding a mutant DNase1L3 protein with at least about an 95% identity with SEQ ID NO: 2-8 for a DNase, with at least one: D38C, A126C, Y261C, N78C, S79C, R80C, K147C, S245C, S91C, S112C, S131C, S253C, S272C, or S279C mutation; and culturing the host cell under conditions such that the mutant DNase1L3 protein is produced by the host cell, wherein the mutant DNase1L3 protein comprises at least one mutation for post-translational modification or attachment of a molecule to the mutant DNase1L3 protein to increase the serum half-life of the mutant DNase1L3 protein.

As embodied and broadly described herein, an aspect of the present disclosure relates to a mutant DNase1L3 protein produced by a method comprising: culturing a bacterial or yeast host cell transformed with an expression vector comprising a DNA sequence comprising the nucleotide sequence encoding the mutant DNase1L3 of SEQ ID NO:2-8, with at least one: D38C, A126C, Y261C, N78C, S79C, R80C, K147C, S245C, S91C, S112C, S131C, S253C, S272C, or S279C mutation, expressing the mutant DNase1L3 in the cultured yeast host cell; and isolating the mutant DNase1L3. In one aspect, the mutant DNase1L3 is post-translationally modified with polyethylene glycol. In another aspect, the mutant DNase1L3 is post-translationally modified with a polyethylene glycol having a molecular mass from 5 kDa to 50 kDa.

As embodied and broadly described herein, an aspect of the present disclosure relates to a process for making a mutant DNase1L3 comprising the steps of: transforming a host cell with a nucleic acid molecule that encodes the mutant DNase1L3 comprising an amino acid sequence of SEQ ID NO: 2-8, with the at least one: D38C, A126C, Y261C, N78C, S79C, R80C, K147C, S245C, S91C, S112C, S131C, S253C, S272C, or S279C mutation; and culturing the host cell under conditions in which the mutant DNase1L3 is produced by the host cell. In one aspect, the host cell comprises E. coli or Pichia pastoris. In another aspect, the host cell produces at least 0.25 mg/L mutant DNase1L3 protein. In another aspect, the host cell produces at least 7.5 mg/L mutant DNase1L3 protein in TB broth.

As embodied and broadly described herein, an aspect of the present disclosure relates to a mutant DNase1L3 made by the process described herein above. In one aspect, the mutant DNase1L3 is post-translationally modified with polyethylene glycol. In another aspect, the mutant DNase1L3 is post-translationally modified with a polyethylene glycol having a molecular mass from 5 kDa to 50 kDa.

As embodied and broadly described herein, an aspect of the present disclosure relates to a method of preventing or treating an autoimmune disease comprising: identifying a subject in need of treatment for the autoimmune disease; and providing an effective amount of a DNase I of SEQ ID NO: 2-8 sufficient to prevent or treat the autoimmune disease. In one aspect, the autoimmune disease is selected from at least one of: systemic lupus erythematosus, autoimmune liver disease, cystic fibrosis, autoimmune hepatitis, primary sclerosing cholangitis, primary biliary cirrhosis, rheumatoid arthritis, systemic sclerosis, scleroderma, asthma, dermatomyositis/polymyositis, autoimmune hemolytic anemia, hepatocellular carcinoma, ovarian cancer, hypocomplementemic urticarial vasculitis syndrome, Behcet's disease, COVID-19, ankylosing spondylitis, obstructive sleep apnea, lung adenocarcinoma, vascular occlusion during severe bacterial infection.

### BRIEF DESCRIPTION OF THE DRAWINGS

For a more complete understanding of the features and advantages of the present invention, reference is now made to the detailed description of the invention along with the accompanying figures and in which:

FIGS. 1A to 1D. The CTD promotes complexed DNA degradation without lipid binding. (FIG. 1A) Plasmid degradation activities for full-length DNase1L3 (D1L3 FL), DNase1L3 ACTD (D1L3 ACTD) and DNase1 (D1) were measured by mixing 200 ng of plasmid DNA with the reported range of DNase concentrations in a 10 μl reaction volume, for 30 min at 37° C. (FIG. 1B) DNase1L3-specific activity was measured using the barrier-to-transfection assay. HEK cells were transfected with 100 ng of eGFP-N1 plasmid after plasmid-lipid complexes were incubated with 100 ng of the indicated DNase at 37° C. for 30 min. Transfection efficiency was measured by flow cytometry. (FIG. 1C, FIG. 1D) DNase1L3-specific activity was measured using immune complex degradation. The indicated concentration of DNase was incubated with chromatin-anti-dsDNA immune complexes, and the remaining anti-dsDNA antibody was measured. The percent immune complex degradation and EC₅₀ were calculated (see McCord, et al., 2022 PMID: 35974043). Graphs represent mean: ±SEM of three independent experiments. **** p<0.0001, *** p<0.005, ** p<0.01, *p<0.05.

FIGS. 2A to 2D. PEGylation of DNase1L3 does not eliminate DNase1L3 activity. (FIG. 2A) DNase1 activity for full-length DNase1 (D1), DNase1L3 (D1L3), DNase1L3 S283X (D1L3 S283X), PEGylated mutant (S112C) DNase1L3 (S112C PEG) or PEGylated mutant (S253C) DNase1L3 (S253C PEG) was measured by mixing 200 ng of plasmid DNA with a range of DNase concentrations for 30 min at 37° C. The EC₅₀ was calculated using logistic modeling. (FIG. 2B) DNase1L3-specific activity was measured using immune complex degradation as in FIG. 1. A range of Dnase concentrations were incubated with chromatin-anti-dsDNA immune complexes, and remaining anti-dsDNA antibody measured. The percent immune complex degradation was calculated. EC₅₀ was determined by logistic modeling. (FIG. 2C) DNase1L3-specific activity was measured using the barrier to transfection assay. HEK cells were transfected with the indicated amounts of eGFP-N1 plasmid after plasmid-lipid complexes were incubated with the indicated DNase at 37° C. for 30 min. Transfection efficiency was measured by flow cytometry. (FIG. 2D) The DNase1L3 Activity Index was calculated by dividing the DNase1 activity (EC₅₀ from A in pmol) by the DNase1L3 activity (EC₅₀ from B in fmol). Since a smaller EC₅₀ represents superior activity, a larger DNase1L3 Activity Index represents improved DNase1L3 activity.

FIGS. 3A to 3E. Mutant DNase1L3 can be PEGylated and purified. The indicated mutations were introduced into human DNase1L3. Human DNase 1L3 was purified and conjugated to either (FIG. 3A) 5 kDa PEG or (FIG. 3B-FIG. 3E) 10 kDa PEG using 1-5 mM TCEP to activate sulfide bonds. Human DNase1L3 was produced using (FIG. 3A-FIG. 3B) p202, or using (FIG. 3C-FIG. 3E) pMATT. pMATT encodes an N-terminal GDITH sequence. (FIG. 3C, FIG. 3D) PEGylated DNase1L3 was separated from non-PEGylated DNase1L3 after PEGylation. Note fractions 13-16 contain PEGylated DNase1L3, while fractions 18-22 contains DNase1L3 that was not PEGylated. Input=PEGylated DNase1L3 D38C. (FIG. 3E) Wild type DNase1L3 can be PEGylated. Wild-type DNase1L3 was expressed using the CyDisco system and PEGylated in the absence or presence of 2-mercaptoethanol. Representative Coomassie blue-stained gels are shown. Mw=molecular weight ladder, TCEP=tris(2-carboxyethyl) phosphine, TEV=Tobacco Etch Virus protease, D1L3=DNase1L3, MBP=maltose-binding protein, PEG=polyethylene-glycol, 2-ME=2-mercaptoethanol.

FIGS. 4A and 4B. FIG. 4A and FIG. 4B show that the CTD does not promote lipid nor microparticle binding: (FIG. 4A) Liposomes or (FIG. 4B) microparticles (MP) were incubated with wild-type DNase1L3 (D1L3FL), DNase1L3 ACTD (D1L3 ACTD), SH3 or SH3-CTD and the supernatants(S) and pellets (P) were prepared in SDS sample buffer. Samples were resolved by SDS-PAGE followed by Coomassie staining (top) or transferred to nitrocellulose and probed with anti-DNase1L3 (bottom). Each blot or Coomassie gel is a representative image from four independent experiments. **** p<0.0001, *** p<0.005, ** p<0.01, *p<0.05.

FIGS. 5A to 5C. Serine to Cysteine mutants retain nuclease and immune complex activity. DNase1, wild type DNase1L3, DNase1L3 ACTD or PEGylated mutant DNase1L3 were assayed for (FIG. 5A) DNase1, or (FIG. 5B) DNase1L3 activity as described in FIGS. 1A to 1D and 4A and 4B. Note for EC₅₀, smaller numbers indicate greater activity. (FIG. 5C) The DNase1L3 activity index was calculated by dividing the DNase1 activity by the DNase1L3 activity. By dividing DNase1 by DNase1L3 activity (instead of vice versa), larger numbers on the DNase1L3 activity index indicate greater DNase1L3-specific activity.

FIGS. 6A to 6C. PEGylated DNase1L3 has improved efficacy over non-PEGylated DNase1L3. Wild type DNase1L3, non-PEGylated mutant DNase1L3, or PEGylated mutant DNase1L3 were assayed for (FIG. 6A) DNase1, or (FIG. 6B) DNase1L3 activity as described in FIG. 4. Note for EC₅₀, smaller numbers indicate greater activity. (FIG. 6C) The DNase1L3 activity index was calculated by dividing the DNase1 activity by the DNase1L3 activity. By dividing DNase1 by DNase1L3 activity (instead of vice versa), larger numbers on the DNase1L3 activity index indicate greater DNase1L3-specific activity.

FIG. 7. Site-specific PEGylation maintains superior DNase1L3 activity compared to PEGylated wild-type DNase1L3. PEGylated wild-type DNase1L3, non-PEGylated mutant DNase1L3, or PEGylated mutant DNase1L3 were assayed for DNase1L3 activity as described in FIG. 4. Note for EC₅₀, smaller numbers indicate greater activity.

FIGS. 8A to 8B. PEGylated DNase1L3 persists in mouse serum. (FIG. 8A) Female NZB/W F1 mice were injected with 5.62 μmol wild type DNase1L3 (WT), PEGylated DNase1L3 S253C (PEG) or saline (Buffer). Serum taken 9 days post-injection was analyzed by Western blot for DNase1L3. (FIG. 8B) Ponceau S staining of the blot. The blot shows 2 sets of mice out of 5 mice per group. Input shows the recombinant protein prior to injection.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

The present invention includes novel DNase1L3 mutants for use in therapy. However, one key challenge to developing it as a therapy is its short predicted serum half-life. The present inventors have made key modifications to the protein DNase1L3 that improves its serum half-life without compromising its activity. DNase1L3 is a small (˜33 kDa) protein, which is why it is assumed to have a short serum half-life. One general method to improve half-life is addition of polyethylene glycol (PEGylation), which increases the size of the protein and protects it from degradation. However, PEGylation has never been applied to DNase1L3. Adding PEG to DNase1L3 requires modification of the protein to facilitate the chemical reaction, but the locations on the protein that can accept PEG without destroying protein function are unknown. Using the crystal structure developed by the present inventors, several novel sites on DNase1L3 were identified that can be mutated to accept PEG. These sites do not compromise nuclease activity, but are predicted to enhance the serum half-life of DNase1L3. Enhancing serum half-life is a necessary prerequisite to developing DNase1L3 as a therapy for any disease.

This solution is unique because no one else has previously targeted the amino acids that we have for PEGylation. Targeting these amino acids involves changing them from serine to cysteine (S91C, S112C, S131C, S253C, S272C) because the PEGylation requires cysteine in those locations. PEGylation has not previously been applied to DNase1L3. The inventors used homology modeling to predict solvent-accessible amino acids that would not interfere with the enzyme activity.

With the successful solution of the crystal structure, the inventors have added additional candidate amino acids. The following additional amino acid mutations were selected: D38C, A126C, Y261C, N78C, S79C, R80C, K147C and S245C. D38C, A126C are homologous to N-linked glycosylation sites in DNase1. PEGylated, five mutants were shown to retain DNase1L3 activity. Y261C is a pre-existing DNase1L3 SNP that does not reduce activity (see Ueki 2014). The mutations N78C, S79C, R80C, K147C and S245C were chosen based on the crystal structure.

The present inventors targeted 3 different locations on the protein to mutate the residues to allow for PEGylation. Targeting these amino acids involves changing them from serine to cysteine any one of: S91C, S112C, and/or S253C because the PEGylation requires cysteine in those locations. PEGylation has not previously been applied to DNase1L3. The present inventors used homology modeling to predict solvent-accessible amino acids that would not interfere with the enzyme activity. Targeted amino acids can also involve changing from serine to cysteine at S131C and/or S272C.

Target serines highlighted in bold and underlined, and numbered by full length DNase1L3. Homo sapiens. Wild-type DNase1L3 Primary Amino Acid sequence (amino acids 21-305 of the long isoform):

(SEQ ID NO: 1)
MRICSFNVRSFGESKQEDKNAMDVIVKVIKRCDIILVMEIKDSNNRICPI
LMEKLNRNSRRGITYNYVISSRLGRNTYKEQYAFLYKEKLVSVKRSYHYH
DYQDGDADVFSREPFVVWFQSPHTAVKDFVIIPLHTTPETSVKEIDELVE
VYTDVKHRWKAENFIFMGDFNAGCSYVPKKAWKNIRLRTDPRFVWLIGDQ
EDTTVKKSTNCAYDRIVLRGQEIVSSVVPKSNSVFDFQKAYKLTEEEALD
VSDHFPVEFKLQSSRAFTNSKKSVTLRKKTKSKRS

The human full length DNase1L3. Homo sapiens, can be modified with one or more mutations selected from: D38C, A126C, Y261C, N78C, S79C, R80C, K147C, or S245C. The DNase1L3 can be modified with 2, 3, 4, 5, 6, 7 or 8 of the mutations. Further mutations can be selected from those listed hereinbelow.

Mutant DNase1L3 of the present invention, mutation at S91C

(SEQ ID NO: 2)
MRICSFNVRSFGESKQEDKNAMDVIVKVIKRCDIILVMEIKDSNNRICPI
LMEKLNRNSRRGITYNYVISCRLGRNTYKEQYAFLYKEKLVSVKRSYHYH
DYQDGDADVFSREPFVVWFQSPHTAVKDFVIIPLHTTPETSVKEIDELVE
VYTDVKHRWKAENFIFMGDFNAGCSYVPKKAWKNIRLRTDPRFVWLIGDQ
EDTTVKKSTNCAYDRIVLRGQEIVSSVVPKSNSVFDFQKAYKLTEEEALD
VSDHFPVEFKLQSSRAFTNSKKSVTLRKKTKSKRS.

Mutant DNase1L3 of the present invention, mutation at S112C

(SEQ ID NO: 3)
MRICSFNVRSFGESKQEDKNAMDVIVKVIKRCDIILVMEIKDSNNRICPI
LMEKLNRNSRRGITYNYVISSRLGRNTYKEQYAFLYKEKLVCVKRSYHYH
DYQDGDADVFSREPFVVWFQSPHTAVKDFVIIPLHTTPETSVKEIDELVE
VYTDVKHRWKAENFIFMGDFNAGCSYVPKKAWKNIRLRTDPRFVWLIGDQ
EDTTVKKSTNCAYDRIVLRGQEIVSSVVPKSNSVFDFQKAYKLTEEEALD
VSDHFPVEFKLQSSRAFTNSKKSVTLRKKTKSKRS.

Mutant DNase1L3 of the present invention, mutation at S253C

(SEQ ID NO: 4)
MRICSFNVRSFGESKQEDKNAMDVIVKVIKRCDIILVMEIKDSNNRICPI
LMEKLNRNSRRGITYNYVISSRLGRNTYKEQYAFLYKEKLVSVKRSYHYH
DYQDGDADVFSREPFVVWFQSPHTAVKDFVIIPLHTTPETSVKEIDELVE
VYTDVKHRWKAENFIFMGDFNAGCSYVPKKAWKNIRLRTDPRFVWLIGDQ
EDTTVKKSTNCAYDRIVLRGQEIVSSVVPKSNCVFDFQKAYKLTEEEALD
VSDHFPVEFKLQSSRAFTNSKKSVTLRKKTKSKRS.

Mutant DNase1L3 of the present invention, mutation at S91C and S112C

(SEQ ID NO: 5)
MRICSFNVRSFGESKQEDKNAMDVIVKVIKRCDIILVMEIKDSNNRICPI
LMEKLNRNSRRGITYNYVISCRLGRNTYKEQYAFLYKEKLVCVKRSYHYH
DYQDGDADVFSREPFVVWFQSPHTAVKDFVIIPLHTTPETSVKEIDELVE
VYTDVKHRWKAENFIFMGDFNAGCSYVPKKAWKNIRLRTDPRFVWLIGDQ
EDTTVKKSTNCAYDRIVLRGQEIVSSVVPKSNSVFDFQKAYKLTEEEALD
VSDHFPVEFKLQSSRAFTNSKKSVTLRKKTKSKRS.

Mutant DNase1L3 of the present invention, mutation at S91C and S253C

(SEQ ID NO: 6)
MRICSFNVRSFGESKQEDKNAMDVIVKVIKRCDIILVMEIKDSNNRICPI
LMEKLNRNSRRGITYNYVISCRLGRNTYKEQYAFLYKEKLVSVKRSYHYH
DYQDGDADVFSREPFVVWFQSPHTAVKDFVIIPLHTTPETSVKEIDELVE
VYTDVKHRWKAENFIFMGDFNAGCSYVPKKAWKNIRLRTDPRFVWLIGDQ
EDTTVKKSTNCAYDRIVLRGQEIVSSVVPKSNCVFDFQKAYKLTEEEALD
VSDHFPVEFKLQSSRAFTNSKKSVTLRKKTKSKRS.

Mutant DNase1L3 of the present invention, mutation at S112C and S253C

(SEQ ID NO: 7)
MRICSFNVRSFGESKQEDKNAMDVIVKVIKRCDIILVMEIKDSNNRICPI
LMEKLNRNSRRGITYNYVISSRLGRNTYKEQYAFLYKEKLVCVKRSYHYH
DYQDGDADVFSREPFVVWFQSPHTAVKDFVIIPLHTTPETSVKEIDELVE
VYTDVKHRWKAENFIFMGDFNAGCSYVPKKAWKNIRLRTDPRFVWLIGDQ
EDTTVKKSTNCAYDRIVLRGQEIVSSVVPKSNCVFDFQKAYKLTEEEALD
VSDHFPVEFKLQSSRAFTNSKKSVTLRKKTKSKRS.

Mutant DNase1L3 of the present invention, mutation at S91C, S112C, and S253C

(SEQ ID NO: 8)
MRICSFNVRSFGESKQEDKNAMDVIVKVIKRCDIILVMEIKDSNNRICPI
LMEKLNRNSRRGITYNYVISCRLGRNTYKEQYAFLYKEKLVCVKRSYHYH
DYQDGDADVFSREPFVVWFQSPHTAVKDFVIIPLHTTPETSVKEIDELVE
VYTDVKHRWKAENFIFMGDFNAGCSYVPKKAWKNIRLRTDPRFVWLIGDQ
EDTTVKKSTNCAYDRIVLRGQEIVSSVVPKSNCVFDFQKAYKLTEEEALD
VSDHFPVEFKLQSSRAFTNSKKSVTLRKKTKSKRS.

Additional variable peptide sequences N-terminal to DNase1L3 due to protease cleavage may also be present.

Example Primers used to mutate serines to cysteines in DNase1L3:

S91C
(SEQ ID NO: 9)
5′- CGT ACA ACT ATG TGA TTA GCT GTC GGC TTG GAA
GAA ACA C -3′
(SEQ ID NO: 10)
5′- GTG TTT CTT CCA AGC CGA CAG CTA ATC ACA TAG
TTG TAC G -3′
S112C
(SEQ ID NO: 11)
5′- CTA CAA GGA AAA GCT GGT GTG TGT GAA GAG GAG
TTA TC -3′
(SEQ ID NO: 12)
5′- GAT AAC TCC TCT TCA CAC ACA CCA GCT TTT CCT
TGT AG -3′
S253C
(SEQ ID NO: 13)
5′- GTT CCC AAG TCA AAC TGT GTT TTT GAC TTC CAG
AAA GC -3′
(SEQ ID NO: 14)
5′- GCT TTC TGG AAG TCA AAA ACA CAG TTT GAC TTG
GGA AC -3′

Method. Mutations were introduced into DNase1L3 by Quickchange mutagenesis: 10-30 ng of DNase 1L3 was mixed with 200 nM of each primer, 300 μM dNTP, and 1 U Verity Pfu in 1×Verity PCR buffer (catalog #31-5020, Tonbo Biosciences, San Diego CA). PCR was run on a BioRad T100 thermal cycler using the following program: (Lid 104 C), 1.95° C. 1 min, 2.95° C. 30 sec, 3.55° C. 1 min, 4.68° C. 12 min, 5. Go to 2, repeat 18 times, 6.68° C. 15 min, 7.4° C. forever.

At step 7, PCR product was removed from the PCR machine, optionally cleaned using the Wizard SV Gel and PCR Clean-up System (catalog #A9282 Promega, Madison, WI) according to manufacturer's instructions.

The PCR product was DpnI digested with the addition of Cutsmart buffer and 20 U DpnI (catalog #R0176 New England Biolabs, Ipswich, MA), incubation at 37° C. for 1 h.

The Dpn-digest was transformed into subcloning efficiency DH5α bacterial cells (catalog #18265017 Invitrogen, Waltham, MA). DNA and bacteria were incubated on ice for 20 min, heat shocked at 37° C. for 20 seconds, and incubated on ice for 2 min. To the bacteria, 0.95 mL LB was added. Bacteria were placed on a shaker at 225×rpm for 1 h at 37° C., then centrifuged 10,000×g for 5 min at room temp. 0.9 mL LB was removed. Bacteria were resuspended in the remaining 100 μL and plated overnight at 37° C. on LB agar with Kanamycin. Colonies were inoculated into 2 mL LB broth, shaken at 225×rpm 37° C. overnight. DNA was extracted from overnight cultures using miniprep kit (catalog #: 2160250, Epoch Life science, Sugar Land, TX) per manufacturer's instructions and sequenced by Sanger sequencing at Genewiz (New Brunswick, NJ). Once mutants were confirmed by sequencing, they were transformed into Rosetta-gami or BL21 cells. Induction and purification was carried out as described in paragraph [0035].

After purification, DNase1L3 was conjugated to PEG using the following method:

1) a) If Protein solution was eluted from size exclusion column in a buffer containing dithiothreitol (DTT):
Concentrate protein to less than or equal to 2.5 ml
Using PD-10 G25 desalting column
   Degas 1St buffer (300 mM NaCl, 1 mM CaCl₂), 20 mM HEPES pH 7.4) for 10 min in vacuum chamber set to 635 mm Hg.
   Equilibrate column with 25 ml of 1^{st} buffer.
   Discard flow through.
   Add sample (purified protein at a volume of 2.5 ml-if needed add more of 1^{st} buffer to make up volume to 2.5 ml).
   Save flow through.
   Elute with 3.5 ml of degassed Elution buffer (300 mM, 1 mM CaCl₂), 20 mM HEPES pH 7.4, 1 mM Tris(2-carboxyethyl) phosphine (TCEP)).
   Collect elution in fractions of 0.3 ml.
   Measure A280 using nanodrop and save fractions corresponding to peak
b) If Protein is not in a solution containing DTT:
   Add Tris(2-carboxyethyl) phosphine (TCEP) to DNase1L3 protein solution to final concentration: 300 mM NaCl, 1 mM CaCl₂), 20 mM HEPES pH 7.4, 1 mM TCEP (which can be varied).
   Degas protein solution for 10 min in vacuum chamber set to 635 mmHg

2) Ensure protein is concentrated to at least 0.05 mg/mL. Incubate TCEP with the protein for at least 10 min at 4° C. before adding maleimide.

3) Calculate protein concentration in μM for the saved fractions using the formula: [DNase1L3](μM)=(μg/ml)/(MW in kDa). In some cases, the amount of TCEP may be varied for some of the PEGylations. The concentration in μg/ml is determined by dividing the absorption at 280 nm (A280) by the extinction coefficient. The extinction coefficient of DNase1L3 is 1.195. MW in KDa is 36. Therefore, the molarity calculation is [DNase1L3](μM)=A280/43.02.

4) Determine the volume of PEG-maleimide to add, using the following formula:
Volume of PEG-maleimide=[PEG-maleimide]final*(Volume of DNase1L3)/[PEG-maleimide]stock
Prepare the PEG-maleimide stock solution at 1 mM to 10 mM. [PEG-maleimide]final is determined as follows:
[PEG-maleimide]final=6*(#mutated Cys)*[DNase1L3]. For PEG 5K maleimide use MW=5000 g/mol

5) Incubate PEG-maleimide and DNase1L3 overnight at 4° C. Assess PEGylation by SDS-PAGE.

The modified DNase1L3 of the present invention. PEGylated DNase1L3 is a potent endonuclease. The DNase1L3 can be affinity purified with Nickel NTA agarose, then cleaved with Tobacco Etch Virus protease (TEV protease). Cleaved DNase1L3 can be further purified by anion exchange and then by size exclusion chromatography. Fractions from the final size exclusion purification can be resolved by SDS-PAGE and Coomassie stained. Purified DNase 1L3 can be resolved by SDS-PAGE, transferred to nitrocellulose and probed with anti-DNase1L3 primary antibody, anti-rabbit HRP secondary antibody and developed with enhanced chemiluminescence. Recombinant, purified DNase1L3 can be incubated at increasing dilutions with 200 ng plasmid DNA for 30 min at 37° C. in 200 mM Tris pH 7.4, 50 mM MgCl₂, 20 mM CaCl₂) with or without 100 μM DNase1L3. Remaining plasmid DNA was resolved on a 1% agarose gel. Purified DNase1L3 can be conjugated to PEG. Unconjugated DNase1L3, PEGylated DNase1L3 (PEG-D1L3), or free PEG can be resolved by SDS-PAGE and visualized with Coomassie Blue. A 36 kDa band that increases in size to 41 kDa is indicative of successful PEGylation. PEGylated DNase1L3 can be incubated at increasing dilutions with 200 ng plasmid DNA for 30 min at 37° C. in 200 mM Tris pH 7.4, 50 mM MgCl₂, and 20 mM CaCl₂). Remaining plasmid DNA can be resolved on a 1% agarose gel. The absence of DNA indicates degradation (see, Shi et al 2017, PMID: 28533778).

### Purification Protocol

### A. Induction

1. Transform competent Rosetta-gami cells with DNase1L3 in p202:
a. Thaw competent Rosetta-gami cells from −80° C. on ice.
b. Mix 1 μl of plasmid DNA (20-40 pg) with 50 μl of bacterial cells in a 1.5 ml tube.
c. Incubate the mixture on ice for 20 minutes.
d. Heat-shock the mixture at 42° C. in water bath for 45 sec.
e. Put the tube back on ice for 2 min.
f. Add 250 μl SOC or LB broth, without antibiotic, to the 1.5 ml tube and shake at 250 rpm at 37° C. for 1 hour.
g. Place LB agar plate containing 50 μg/mL Kanamycin in incubator alongside mixture to warm up the plate from 4° C.
h. Spread contents of the 1.5 ml tube on the agar plate and incubate at 37° C. overnight.

2. Sample multiple colonies and add them to 150 ml LB (with 150 μl 50 mg/ml Kanamycin) in an autoclaved 250 ml culture flask with baffles.

3. Shake flask at 250 rpm at 37° C. for 20 hours to prepare the overnight culture.

4. Transfer 20 ml overnight culture to each of 6 autoclaved 1-liter culture flasks containing 1 L of terrific broth (TB) supplemented with 0.1 M potassium phosphate buffer. Potassium phosphate buffer can be formulated directly in TB or add 100 ml 1 M potassium phosphate buffer per liter TB.

5. Add 1 ml 50 mg/ml Kanamycin to each 1-liter culture flasks, then shake at 250 rpm at 37° C. until the OD₆₀₀ reaches 1.5 to 1.8 (typically 7 hours).

6. When the OD₆₀₀ reaches 1.5-1.8, turn temperature down to 18° C., 1 hour later induce each culture with 400 μl of 1 M Isopropyl β-D-1-thiogalactopyranoside (IPTG). Shake at 250 rpm in 18° C. for 10 more hours.

7. Transfer culture flasks to 1 L centrifuge tubes and centrifuge at 6200×g for 15 min at 4° C. to pellet cells.

8. Save pellet, discard supernatant.

9. Transfer pellets to 50 ml screw top tubes, with 2 pellets to a tube, freeze all tubes at −80° C.

### B. Lysis

1. Resuspend frozen pelleted cells from 2 1-liter cultures in 200 ml PMSF Lysis buffer. Use freshly prepared PMSF.
a. PMSF Lysis buffer: 300 mM NaCl, 5 mM CaCl₂, 20 mM HEPES, 1 mM PMSF pH 7.4.

2. Mechanically lyse resuspended cells in a Microfluidizer Processor M-110EH.

3. Spin down at 45,900×g for 45 min at 4° C. in centrifuge.

4. Save supernatant for EITHER C. First Purification Step Using Nickel
Column OR D. First Purification Step Using Amylose Resin.

### C. First Purification Step Using Nickel Column

1. Clean Nickel Column (filled with 30 ml Ni-NTA agarose).
b. Fill Column with 100 ml of 0.1 M Ethylenediaminetetraacetic acid (EDTA) pH 8.
c. Rinse Column with 2 column volumes of dH₂O.

2. Prime NTA-agarose with Nickel
d. Fill Column with 50 ml of 100 mM NiSO₄.
e. Rinse Column with 2 column volumes of dH₂O.

3. Equilibrate NTA-agarose Ni Column
f. Fill Column w/50 ml of Lysis Buffer.

4. Bind Lysate to NTA-agarose Beads
g. Transfer Lysate and beads to large enough beaker.
h. Allow to bind in 4° C. refrigerator for 2 hours.

5. Wash Lysate
i. Pour lysate and beads back into column, gently.
j. Allow lysate to go through filter, collect, save 20 μl fraction for SDS page as “ni col. flowthrough”.
k. Wash column with 100 ml lysis buffer, collect, save 20 μl fraction for SDS page as “ni col. wash”.
l. Wash column with 100 ml 30 mM imidazole buffer, collect, save 20 μl fraction for SDS page as “ni col 30 mM imi”.
   i. 30 mM imidazole buffer: 5 mM Maltose, 1 mM CaCl₂), 150 mM NaCl, 20. mM HEPES, 30 mM imidazole; pH 7.4.

6. Elute column
m. Elute column in 50 ml 250 mM imidazole buffer, collect, save 20 μl fraction for SDS page as “ni col 250 mM imi uncut”.
   i. 250 mM imidazole buffer: 5 mM Maltose, 1 mM CaCl₂), 150 mM NaCl, 20 mM HEPES, 250 mM imidazole; pH 7.4.

7. Add 1 ml 5 mg/ml Tobacco Etch Virus (TEV) protease to eluate and incubate overnight at 4° C. Save 20 μl fraction for SDS page as “ni col 250 mM imi cut with TEV”.

8. Resolve samples reserved above by SDS-PAGE (Mini-proteanTGX Stain-Free Any kD (unique formulation)). Verify presence of DNase1L3 in eluate (˜80 kDa fusion protein, ˜33 kDa cut protein) and proceed to E. S Sepharose column.

### D. First Purification Step Using Amylose Resin.

1. Pour prepared amylose resin (see H) into 100 ml volume column with frit.

2. Wash resin with three column volumes of dH₂O and then one column volume of Lysis buffer.

3. Cap bottom of column, pour lysate over resin, seal, and leave at 4° C. overnight to bind.

4. Wash and elute the column as follows:
a Collect column flowthrough, save 20 μl fraction for SDS page as flowthrough 1.
b. Wash column with 100 ml Lysis buffer, save 20 μl fraction for SDS page as wash 1.
c. Elute column with 75 ml maltose buffer, save 20 μl fraction for SDS page as elution 1.
   i. Maltose buffer: 300 mM NaCl, 1 mM CaCl₂), 40 mM maltose, 20 mM HEPES pH 7.4.

5. Run the flowthrough again with 5 ml of fresh amylose resin by repeating step D.1-3.

6. Repeat elution step D.4 again, but save fractions as (respectively): flowthrough 2, wash 2, and elution 2.

7. Run the flowthrough from step D.6 through used amylose resin from step D.5.

8. Repeat step D.4, saving fractions as (respectively): flowthrough 3, wash 3, and elution 3.

9. Add 1 ml 5 mg/ml Tobacco Etch Virus (TEV) protease to each eluate and incubate overnight at 4° C. In the morning collect 20 μl fraction for SDS page from each elution with TEV protease added as (respectively): elution 1 with TEV, elution 2 with TEV, and elution 3 with TEV.

10. Resolve samples reserved above by SDS-PAGE (Mini-proteanTGX Stain-Free Any kD (unique formulation)). Verify presence of DNase1L3 in each eluate by mass. Combine elutions containing DNase1L3 and proceed to E. S Sepharose column.

### E. S Sepharose Column

1. Combine column eluate containing DNase1L3 and dilute 2:1, eluate: buffer using A buffer.
a. A buffer: 50 mM NaCl, 1 mM CaCl₂), 20 mM HEPES, pH 7.4.

2. Prepare FPLC by turning on UV absorbance detector/emitter and washing sulphopropyl (SP) sepharose column with A buffer (roughly 60 ml).

3. Load diluted Nickel column eluate, collect waste line for SDS page.

4. Run A buffer for 50 ml to bring down [NaCl] in the lines.

5. Run gradient of increasing salt concentration using B buffer for 18 min, and collect 3 ml fractions.
a. B buffer: 1 M NaCl, 1 mM CaCl₂), 20 mM HEPES, pH 7.4.

6. Resolve fractions by SDS-PAGE to determine which UV absorbance peak corresponds to DNase1L3.

7. Pool the fractions that contain DNase1L3.

### F. Size Exclusion Chromatography

1. Prepare the FPLC using the size exclusion chromatography column (Superdex 75) by ensuring the UV absorbance detector is on and washing the column with 200 ml of SEC buffer.
a. SEC buffer: 150 mM NaCl, 1 mM CaCl₂), 20 mM HEPES, pH 7.4.

2. Concentrate to <1 ml the combined fractions from the S Column that contained DNase1L3 as shown by the last gel.
a. Concentrate using centrifugal filter, and centrifuging at 5000×g at 4° C. in 15 ml. increments, mixing in between centrifugation, until volume between 0.8 and 1 ml.

3. Inject the concentrated DNase1L3 into the FPLC loop.

4. Load onto column with SEC buffer, collecting 3 ml fractions 50 ml later.

5. Run fractions on SDS page gel to determine the UV absorbance peak that corresponds to DNase1L3.

6. Save fractions that correspond to DNase1L3 on peak UV absorbance and SDS-PAGE.

### G. Buffers and Solutions

Potassium Phosphate Buffer: 23.1 g KH₂PO₄ and 125.4 g K₂HPO₄ per 1 L.

Terrific Broth: Tryptone 12 g, Yeast Extract 24 g, 50% b/v glycerol 8 ml, 892 ml water; per 1 L LB Broth: Tryptone 10 g, Yeast Extract 5 g, NaCl 10 g.

LB Agar Plates: Tryptone 10 g, Yeast Extract 5 g, NaCl 10 g, Agar 15 g; per 1 L

PMSF Lysis buffer: 300 mM NaCl, 5 mM CaCl₂), 1 mM PMSF, 20 mM HEPES, pH 7.4 Difco LB Broth, Miller: 12.5 g of LB Media per 500 ml.

Lysis buffer: 5 mM Maltose, 150 mM NaCl, 1 mM CaCl₂), 20 mM HEPES, pH 7.4.

Maltose buffer: 300 mM NaCl, 1 mM CaCl₂), 40 mM maltose, 20 mM HEPES pH 7.4.

A buffer: 50 mM NaCl, 1 mM CaCl₂), 20 mM HEPES, pH 7.4.

B buffer: 1 M NaCl, 1 mM CaCl₂), 20 mM HEPES, pH 7.4.

SEC buffer: 300 mM NaCl, 1 mM CaCl₂), 20 mM HEPES, pH 7.4.

30 mM imidazole buffer: 5 mM Maltose, 1 mM CaCl₂), 150 mM NaCl, 20 mM HEPES, 30 mM imidazole; pH 7.4.

250 mM imidazole buffer: 5 mM Maltose, 1 mM CaCl₂), 150 mM NaCl, 20 mM HEPES, 250 mM imidazole; pH 7.4.

Amylose Prep Solution: 0.5 M NaCl, 50 mM glycine-HCL; pH 2.0.

### H. Amylose Resin Preparation

1. Evenly suspend 10 g amylose (Sigma, grade III from potato) in 40 ml water. Warm suspension to 50° C. in water bath for 10 min.

2. Add 60 ml 5 M NaOH while stirring rapidly. Then add 30 ml epichlorohydrin while continuing to stir rapidly. This suspension should solidify into a gel within 10 min, when the suspension begins to solidify stop stirring. Allow the gel to cool to room temperature and incubate at room temperature for 45 min.

3. Wash gel with 200 ml of dH₂O and transfer to a blender. Fragment the gel by blending 3-4 times in 7 second intervals at low speed using 200 watt hand blender, in a suspension of water. Wash again in dH₂O.

4. Transfer broken gel to a graduated cylinder and resuspend in 100 ml of Amylose prep solution. Between each wash allow the gel to settle, to separate fine crosslinked amylose particles left in the supernatant. Once settled decant the supernatant by pouring off supernatant, and repeat resuspension then decantation of amylose particles 2 more times for a total of 3 washes.

5. Amylose prep solution: 0.5 M NaCl, 50 mM glycine-HCl, pH 2.0.

6. Resuspend and decant two more times as in step H.4 replacing amylose prep solution with dH₂O.

7. Can be stored in 20% ethanol.

DNase1 Assay. DNase1 assays were performed as described (Shi, G., Abbott, K. N., Wu, W., Salter, R. D. & Keyel, P. A. DNase1L3 regulates inflammasome-dependent cytokine secretion. Front. Immunol. 8, DOI: 10.3389/fimmu.2017.00522 (2017)) 200 ng plasmid DNA was incubated with varying concentrations of DNase1L3 full length, DNase1L3 DCTD or DNase1 in DNase assay buffer (20 mM Tris, pH 7.4, 5 mM MgCl₂, 2 mM CaCl₂)) for 30 min at 37° C. The extent of DNA degradation was quantitated by measuring the integrated intensity of degraded and intact plasmid DNA from Gel Red-stained agarose gels using Photoshop Creative Suite (Adobe, San Jose, CA) and determining the percent degradation. The EC₅₀ for DNase1 activity was calculated from the dose-response curve using logistic modeling.

Barrier to Transfection. HEK cells were plated one day prior to the assay at 5×10⁵ cells per well in a 24 well plate. DNA-lipid complexes were prepared by incubating 25-100 ng of eGFP-N1 plasmid (Takara Biosciences) with Lipofectamine 2000 for 20 min. DNA-lipid complexes were then incubated with full length DNase1L3, DNase1L3 DCTD or DNase1 at 37° C., 5% CO₂ for 30 minutes. HEK cells were then transfected with the control or the DNase treated DNA-lipid complexes and incubated for 48 h. The cells were supplemented with fresh D10 media after 24 h. Cells were harvested, washed in FACS buffer (2% Fetal calf serum, 0.05% NaN₃ in 1×PBS) and analyzed on an Attune N×T flow cytometer. Transfection efficiency was 70% in control cells. Reduced transfection efficiency was calculated compared to control transfected cells.

Immune Complex Degradation. To measure immune complex degradation, a modified ELISA protocol was used. ELISA plates were pre-coated with 0.05 mg/mL poly-L-lysine at room temp for 20 min, washed with 1× nuclease free water, and coated with 5 μg/ml calf thymus DNA (Sigma) overnight at 4° C. After washing 3× in PBS with 0.05% Tween (PBST) and blocking for 1 h at room temp with 1% BSA in PBST, 250 μg/mL anti-dsDNA antibody was added to all wells except the standard curve. The standard curve received 2-fold dilutions of anti-dsDNA antibody starting at 500 μg/mL. After 1 h, plates were washed 3× in PBST, DNase (diluted into 20 mM HEPES, 300 mM NaCl, 1 μM CaCl₂)) was added and plates incubated 37° C. for 2 h. Plates were washed 3× in PBST, incubated with HRP conjugated goat anti-mouse IgG antibody (1:20,000), and developed using 10 mg/ml TMB (Sigma), 3%

H₂O₂ (Walmart, Fayetteville, AR) in 100 mM sodium acetate, pH 5.5. The reaction was stopped with 0.5 M H₂SO₄. A450 was measured and antibody concentration in each well calculated. The percentage of dsDNA antibody remaining in the well was calculated by comparison to control. Percentage immune complex degradation was 100-% remaining antibody. The EC₅₀ for each DNase was calculated using logistic regression.

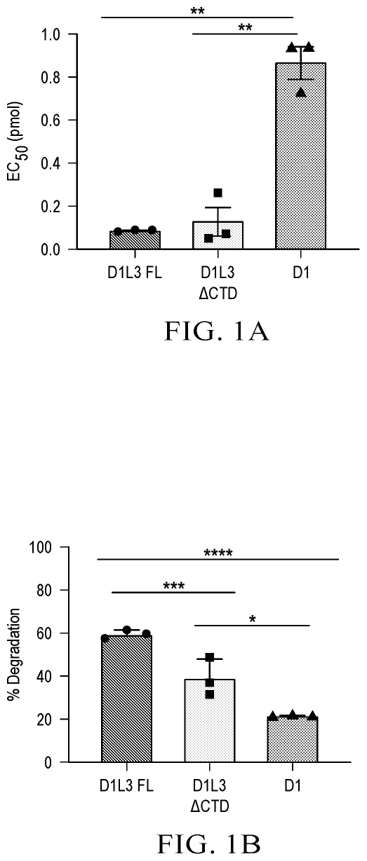

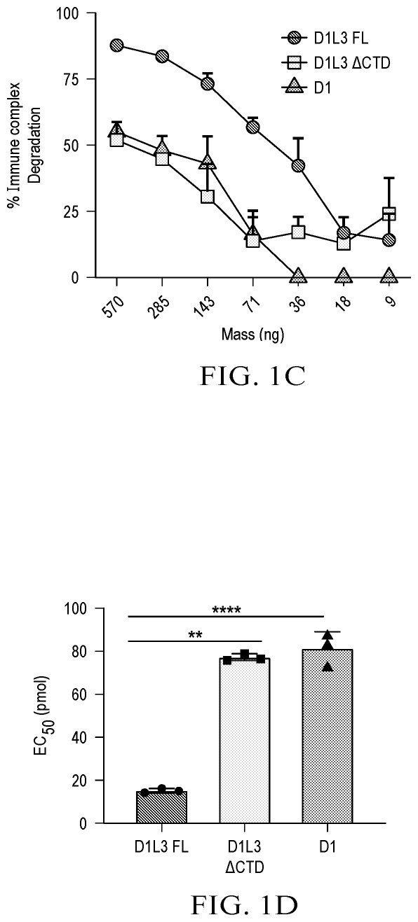

FIGS. 1A to 1D. Deletion of the carboxy-terminal domain (CTD) promotes complexed DNA degradation without lipid binding. (FIG. 1A) DNase1 activity for full-length DNase1L3 (D1L3 FL), DNase1L3 ACTD (D1L3 ACTD), and DNase1 (D1) was measured by mixing 200 ng of plasmid DNA with a range of DNase concentrations for 30 min at 37° C. (FIG. 1B) DNase1L3-specific activity was measured using the barrier-to-transfection assay. HEK cells were transfected with eGFP-N1 plasmid after plasmid-lipid complexes were incubated with the indicated DNase at 37° C. for 30 min. Transfection efficiency was measured by flow cytometry. (FIG. 1C, FIG. 1D) DNase1L3-specific activity was measured using immune complex degradation. The indicated concentration of DNase was incubated with chromatin-anti-dsDNA immune complexes, and the remaining anti-dsDNA antibody was measured. The percent immune complex degradation and EC₅₀ were calculated (see McCord, et al.). Graphs represent mean: SEM of three independent experiments. **** p<0.0001, *** p<0.005, ** p<0.01, *p<0.05.

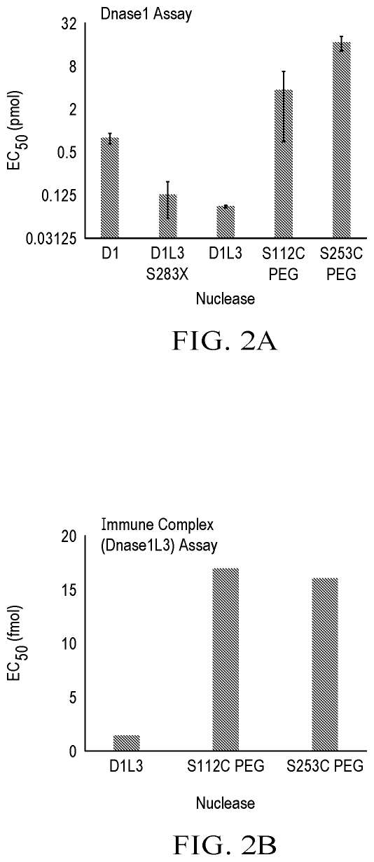

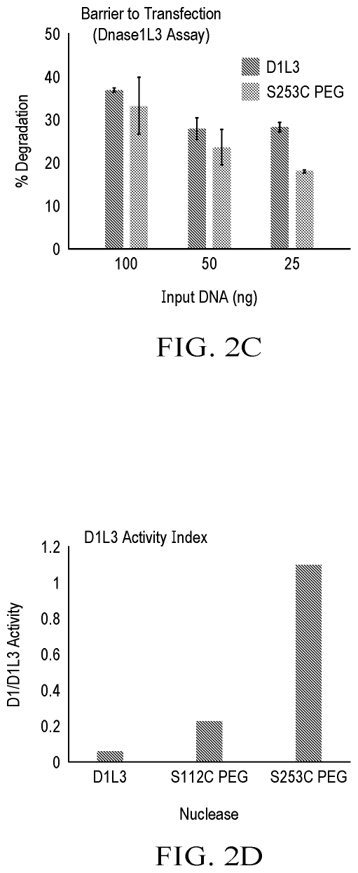

FIGS. 2A to 2D. PEGylation of DNase1L3 does not eliminate DNase1L3 activity. (FIG. 2A) DNase1 activity for full-length DNase1 (D1), DNase1L3 (D1L3), DNase1L3 S283X (D1L3 S283X), PEGylated mutant (S112C) DNase1L3 (S112C PEG) or PEGylated mutant (S253C) DNase1L3 (S253C PEG) was measured by mixing 200 ng of plasmid DNA with a range of DNase concentrations for 30 min at 37° C. The EC₅₀ was calculated using logistic modeling. (FIG. 2B) DNase1L3-specific activity was measured using immune complex degradation as in FIG. 1. A range of Dnase concentrations were incubated with chromatin-anti-dsDNA immune complexes, and remaining anti-dsDNA antibody measured. The percent immune complex degradation was calculated. EC₅₀ was determined by logistic modeling. (FIG. 2C) DNase1L3-specific activity was measured using the barrier to transfection assay. HEK cells were transfected with the indicated amounts of eGFP-N1 plasmid after plasmid-lipid complexes were incubated with the indicated DNase at 37° C. for 30 min. Transfection efficiency was measured by flow cytometry. (FIG. 2D) The DNase1L3 Activity Index was calculated by dividing the DNase1 activity (EC₅₀ from A in μmol) by the DNase1L3 activity (EC₅₀ from B in fmol). Since a smaller EC₅₀ represents superior activity, a larger DNase1L3 Activity Index represents improved DNase1L3 activity.

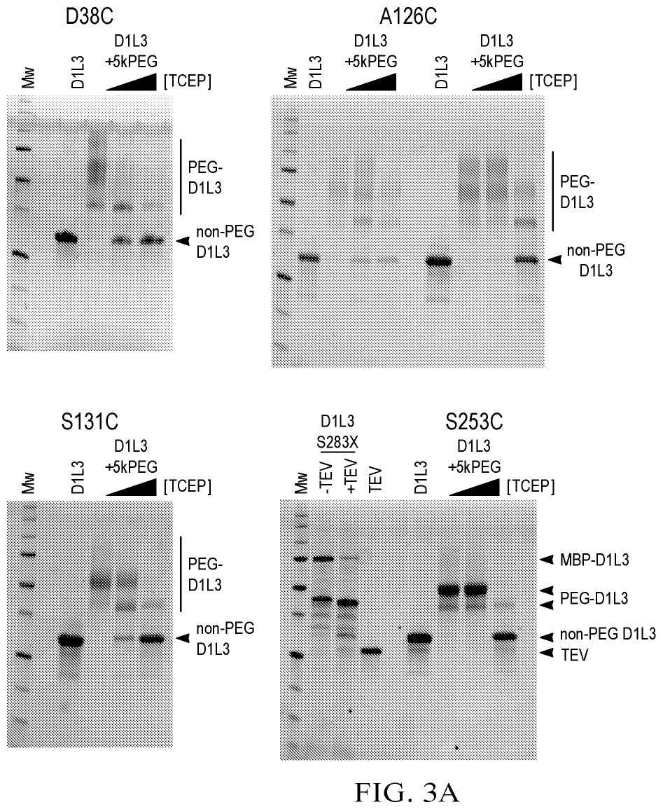

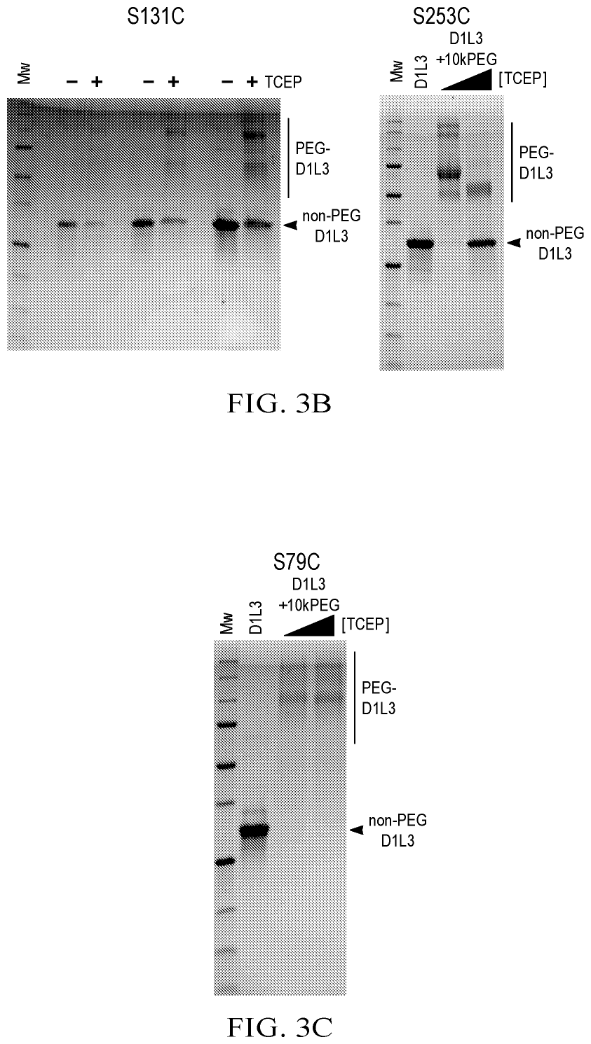

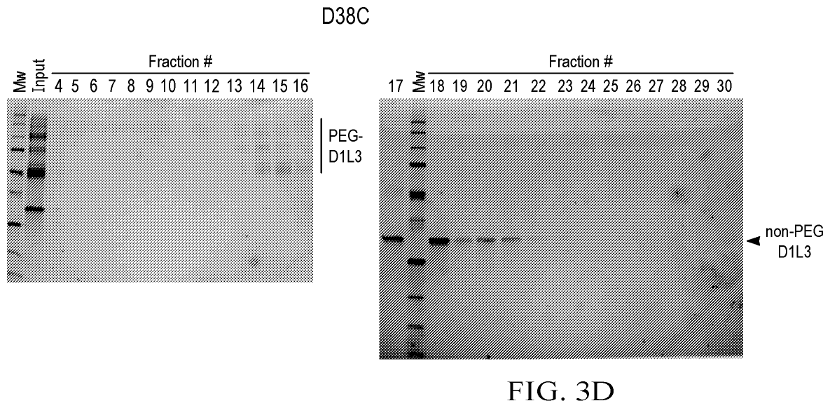

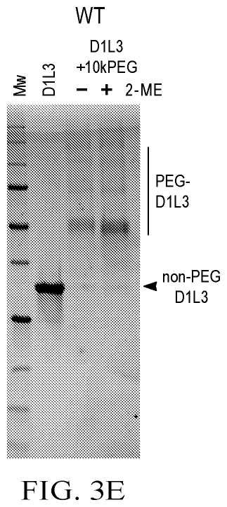

FIGS. 3A to 3E. Mutant DNase1L3 can be PEGylated and purified. The indicated mutations were introduced into human DNase1L3. Human DNase1L3 was purified and conjugated to either (FIG. 3A) 5 kDa PEG or (FIG. 3B-FIG. 3E) 10 kDa PEG using 1-5 mM TCEP to activate sulfide bonds. Human DNase1L3 was produced using (FIG. 3A-FIG. 3B) p202, or using (FIG. 3C-FIG. 3E) pMATT. pMATT encodes an N-terminal GDITH sequence. (FIG. 3C, FIG. 3D) PEGylated DNase1L3 was separated from non-PEGylated DNase1L3 after PEGylation. Note fractions 13-16 contain PEGylated DNase1L3, while fractions 18-22 contains DNase1L3 that was not PEGylated. Input=PEGylated DNase1L3 D38C. (FIG. 3E) Wild type DNase1L3 can be PEGylated. Wild type DNase1L3 was expressed using the CyDisco system and PEGylated in the absence or presence of 2-mercaptoethanol. Representative Coomassie blue-stained gels are shown. Mw=molecular weight ladder, TCEP=tris(2-carboxyethyl) phosphine, TEV=Tobacco Etch Virus protease, D1L3=DNase1L3, MBP=maltose-binding protein, PEG=polyethylene-glycol, 2-ME=2-mercaptoethanol.

### Assay.

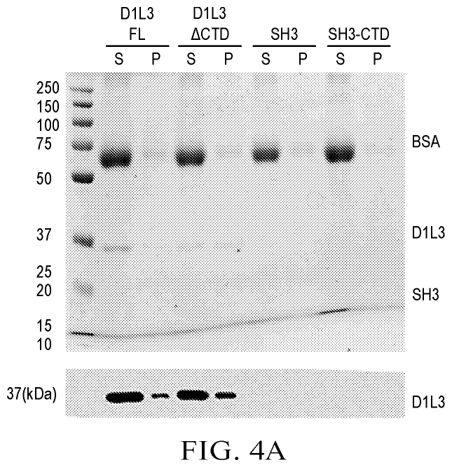

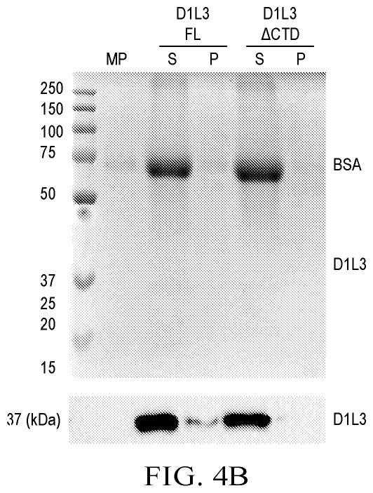

FIGS. 4A and 4B show that the CTD does not promote lipid nor microparticle binding: (FIG. 1A) Liposomes or (FIG. 1B) microparticles (MP) were incubated with wild-type DNase1L3 (D1L3FL), DNase1L3 ACTD (D1L3 ACTD), SH3 or SH3-CTD and the supernatants(S) and pellets (P) were prepared in SDS sample buffer. Samples were resolved by SDS-PAGE followed by Coomassie staining (top) or transferred to nitrocellulose and probed with anti-DNase1L3 (bottom). Each blot or Coomassie gel is a representative image from four independent experiments. **** p<0.0001, *** p<0.005, ** p<0.01, *p<0.05.

Lead Identification Via DNase1 and DNase1L3 Assay.

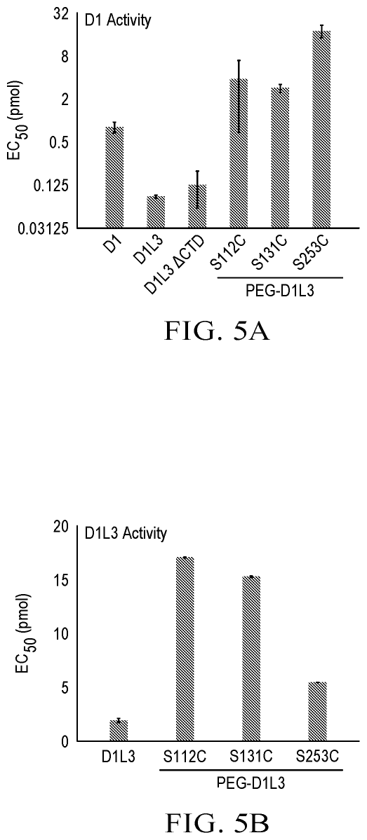

FIGS. 5A to 5C. Serine to Cysteine mutants retain nuclease and immune complex activity. DNase1, wild type DNase1L3, DNase1L3 ACTD or PEGylated mutant DNase1L3 were assayed for (FIG. 5A) DNase1, or (FIG. 5B) DNase1L3 activity as described in FIGS. 1A to 1D, and 4A and 4B. Note for EC₅₀, smaller numbers indicate greater activity. (FIG. 5C) The DNase1L3 activity index was calculated by dividing the DNase1 activity by the DNase1L3 activity. By dividing DNase1 by DNase1L3 activity (instead of vice versa), larger numbers on the DNase1L3 activity index indicate greater DNase1L3-specific activity.

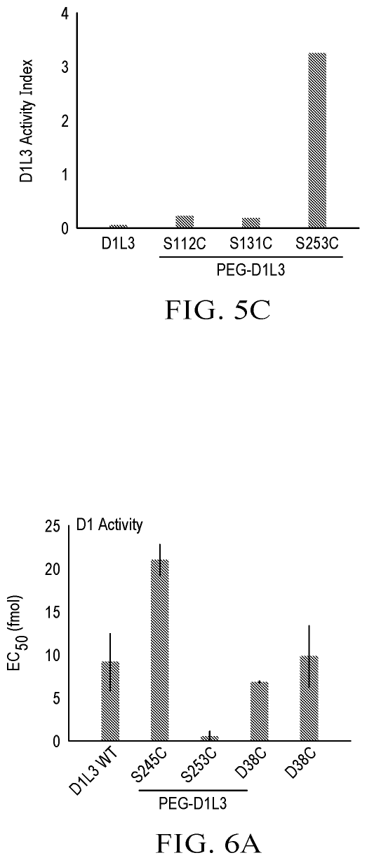

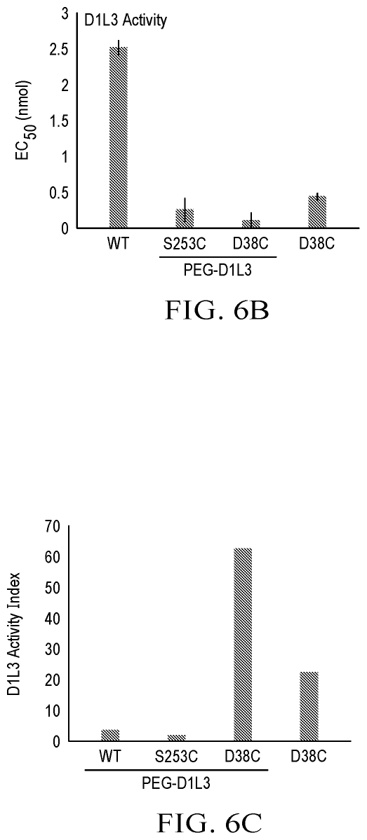

FIGS. 6A to 6C. PEGylated DNase1L3 has improved efficacy over non-PEGylated DNase1L3. Wild type DNase1L3, non-PEGylated mutant DNase1L3, or PEGylated mutant DNase1L3 were assayed for (FIG. 6A) DNase1, or (FIG. 6B) DNase1L3 activity as described in FIG. 4. Note for EC₅₀, smaller numbers indicate greater activity. (FIG. 6C) The DNase1L3 activity index was calculated by dividing the DNase1 activity by the DNase1L3 activity. By dividing DNase1 by DNase1L3 activity (instead of vice versa), larger numbers on the DNase1L3 activity index indicate greater DNase1L3-specific activity.

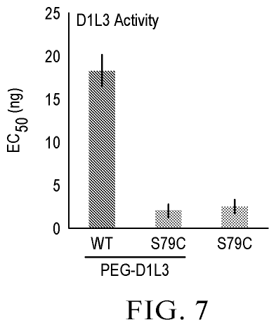

FIG. 7. Site-specific PEGylation maintains superior DNase1L3 activity compared to PEGylated wild-type DNase1L3. PEGylated wild-type DNase1L3, non-PEGylated mutant DNase1L3, or PEGylated mutant DNase1L3 were assayed for DNase1L3 activity as described in FIG. 4. Note for EC₅₀, smaller numbers indicate greater activity.

### Animal Studies.

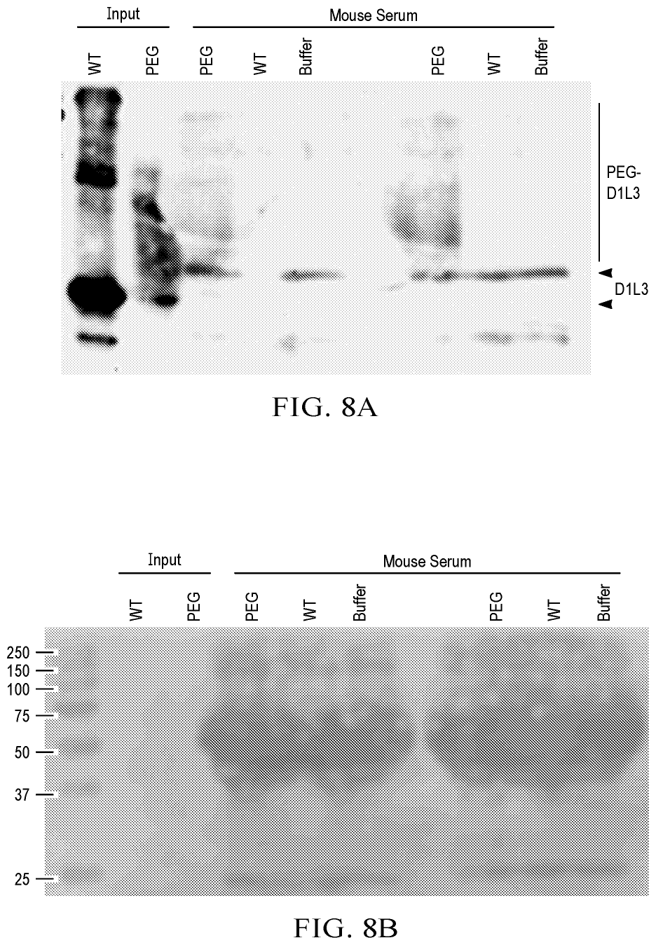

FIGS. 8A to 8B. PEGylated DNase1L3 persists in mouse serum. (FIG. 8A) Female NZB/W F1 mice were injected with 5.62 μmol wild type DNase1L3 (WT), PEGylated DNase1L3 S253C (PEG) or saline (Buffer). Serum taken 9 days post-injection was analyzed by Western blot for DNase1L3. (FIG. 8B) Ponceau S staining of the blot. The blot shows 2 sets of mice out of 5 mice per group. Input shows the recombinant protein prior to injection.

As embodied and broadly described herein, an aspect of the present disclosure relates to an isolated and purified nucleic acid comprising, consisting essentially of, or consisting of, a nucleic acid encoding a mutant DNase1L3 comprising at least one mutation for post-translational attachment of polyethylene-glycol (PEG) to the mutant DNase1L3 to increase the serum half-life of the mutant DNase1L3. In one aspect, the nucleic acid further comprises a nucleic acid sequence optimized for microbial expression. In another aspect, the mutant DNase1L3 comprises the mutant DNase1L3 comprises at least one of: D38C, A126C, Y261C, N78C, S79C, R80C, K147C, or S245C mutation. In another aspect, the mutant DNase 1L3 further comprises at least one of: S91C, S112C, S131C, S253C, S272C, or S279C mutation. In another aspect, the mutant DNase1L3 comprises the mutant DNase1L3 comprises at least two mutations selected from D38C, A126C, Y261C, N78C, S79C, R80C, K147C, S245C, S91C, S112C, S131C, S253C, S272C, or S279C mutations. In another aspect, the nucleic acid further comprises a nucleic acid segment encoding a leader sequence. In another aspect, the nucleic acid further comprises a codon-optimized mutant DNase1L3 nucleic acid encoding SEQ ID NOS: 2-8, with the at least one: D38C, A126C, Y261C, N78C, S79C, R80C, K147C, S245C, S91C, S112C, S131C, S253C, S272C, or S279C mutation. In another aspect, the nucleic acid encodes a protein that comprises an about 95, 96, 97, 98, 99, or 100 percent identity or higher with a codon-optimized mutant DNase1L3 of SEQ ID NO: 2-8, with the at least one: D38C, A126C, Y261C, N78C, S79C, R80C, K147C, S245C, S91C, S112C, S131C, S253C, S272C, or S279C mutation.

As embodied and broadly described herein, an aspect of the present disclosure relates to an expression vector comprising, consisting essentially of, or consisting of, a nucleic acid encoding the protein of SEQ ID NO: 2-8 for a mutant DNase1L3 comprising at least one mutation for post-translational attachment of polyethylene-glycol (PEG) operably linked to a promoter recognized by a host cell transformed with the vector. In one aspect, the host cell is a bacterial or yeast cell. In another aspect, the host cell comprises E. coli or Pichia pastoris.

As embodied and broadly described herein, an aspect of the present disclosure relates to a mutant DNase1L3 having at least about a 95% identity with a nucleic acid sequence encoding a mutant DNase1L3 comprising, consisting essentially of, or consisting of, at least one mutation for post-translational attachment of polyethylene-glycol (PEG) to the mutant DNase1L3 to increase the serum half-life of the mutant DNase1L3. In one aspect, the mutant DNase1L3 further comprises a nucleic acid sequence optimized for microbial expression. In another aspect, the mutant DNase1L3 comprises at least one of: D38C, A126C, Y261C, N78C, S79C, R80C, K147C, or S245C mutation. In another aspect, the mutant DNase1L3 further comprises at least one of: S91C, S112C, S131C, S253C, S272C, or S279C mutation. In another aspect, the mutant DNase1L3 comprises the mutant DNase1L3 comprises at least two mutations selected from D38C, A126C, Y261C, N78C, S79C, R80C, K147C, S245C, S91C, S112C, S131C, S253C, S272C, or S279C mutations. In another aspect, the nucleic acid further comprises a nucleic acid segment encoding a leader sequence. In another aspect, the nucleic acid further comprises a codon-optimized mutant DNase1L3 nucleic acid encoding SEQ ID NOS: 2-8, with at least one: D38C, A126C, Y261C, N78C, S79C, R80C, K147C, S245C, S91C, S112C, S131C, S253C, S272C, or S279C mutation. In another aspect, the nucleic acid encodes a mutant protein that comprises an about 95, 96, 97, 98, 99, or 100 percent identity or higher with a codon-optimized mutant DNase1L3 of SEQ ID NO: 2-8, with the at least one: D38C, A126C, Y261C, N78C, S79C, R80C, K147C, S245C, S91C, S112C, S131C, S253C, S272C, or S279C mutation. In another aspect, the mutant DNase1L3 is post-translationally modified with polyethylene glycol. In another aspect, the mutant DNase1L3 is post-translationally modified with a polyethylene glycol having a molecular mass from 5 kDa to 50 kDa.

As embodied and broadly described herein, an aspect of the present disclosure relates to a host cell transformed with an expression vector comprising, consisting essentially of, or consisting of a nucleic acid encoding an amino acid sequence of SEQ ID NO: 2-8 for a mutant DNase1L3, with the at least one: D38C, A126C, Y261C, N78C, S79C, R80C, K147C, S245C, S91C, S112C, S131C, S253C, S272C, or S279C mutation. In one aspect, the host cell comprises a bacterial or a yeast cell. In another aspect, the host cell comprises E. coli, Pichia pastoris, or host strains that allows enhanced disulfide bond formation and enhanced expression of eukaryotic proteins that contain codons rarely used in E. coli.

As embodied and broadly described herein, an aspect of the present disclosure relates to a process for making a protein with DNase activity comprising, consisting essentially of, or consisting of, the steps of: transforming a host cell with an isolated nucleic acid comprising a nucleotide sequence encoding a mutant DNase1L3 protein with at least about an 95% identity with SEQ ID NO: 2-8 for a DNase, with at least one: D38C, A126C, Y261C, N78C, S79C, R80C, K147C, S245C, S91C, S112C, S131C, S253C, S272C, or S279C mutation; and culturing the host cell under conditions such that the mutant DNase 1L3 protein is produced by the host cell, Wherein the mutant DNase1L3 protein comprises at least one mutation for post-translational modification or attachment of a molecule to the mutant DNase 1L3 protein to increase the serum half-life of the mutant DNase1L3 protein.

As embodied and broadly described herein, an aspect of the present disclosure relates to a mutant DNase1L3 protein produced by a method comprising, consisting essentially of, or consisting of: culturing a bacterial or yeast host cell transformed with an expression vector comprising a DNA sequence comprising the nucleotide sequence encoding the mutant DNase1L3 of SEQ ID NO:2-8, with at least one: D38C, A126C, Y261C, N78C, S79C, R80C, K147C, S245C, S91C, S112C, S131C, S253C, S272C, or S279C mutation, expressing the mutant DNase1L3 in the cultured yeast host cell; and isolating the mutant DNase1L3. In one aspect, the mutant DNase1L3 is post-translationally modified with polyethylene glycol. In another aspect, the mutant DNase1L3 is post-translationally modified with a polyethylene glycol having a molecular mass from 5 kDa to 50 kDa.

As embodied and broadly described herein, an aspect of the present disclosure relates to a process for making a mutant DNase1L3 comprising, consisting essentially of, or consisting of, the steps of: transforming a host cell with a nucleic acid molecule that encodes the mutant DNase1L3 comprising an amino acid sequence of SEQ ID NO: 2-8, with the at least one: D38C, A126C, Y261C, N78C, S79C, R80C, K147C, S245C, S91C, S112C, S131C, S253C, S272C, or S279C mutation; and culturing the host cell under conditions in which the mutant DNase1L3 is produced by the host cell. In one aspect, the host cell comprises E. coli or Pichia pastoris. In another aspect, the host cell produces at least 0.25 mg/L mutant DNase1L3 protein. In another aspect, the host cell produces at least 7.5 mg/L mutant DNase1L3 protein in TB broth.

As embodied and broadly described herein, an aspect of the present disclosure relates to a mutant DNase1L3 made by the process described herein above. In one aspect, the mutant DNase1L3 is post-translationally modified with polyethylene glycol. In another aspect, the mutant DNase1L3 is post-translationally modified with a polyethylene glycol having a molecular mass from 5 kDa to 50 kDa.

As embodied and broadly described herein, an aspect of the present disclosure relates to a method of preventing or treating an autoimmune disease comprising, consisting essentially of, or consisting of: identifying a subject in need of treatment for the autoimmune disease; and providing an effective amount of a DNase I of SEQ ID NO: 2-8 sufficient to prevent or treat the autoimmune disease. In one aspect, the autoimmune disease is selected from at least one of: systemic lupus erythematosus, autoimmune liver disease, cystic fibrosis, autoimmune hepatitis, primary sclerosing cholangitis, primary biliary cirrhosis, rheumatoid arthritis, systemic sclerosis, scleroderma, asthma, dermatomyositis/polymyositis, autoimmune hemolytic anemia, hepatocellular carcinoma, ovarian cancer, hypocomplementemic urticarial vasculitis syndrome, Behcet's disease, COVID-19, ankylosing spondylitis, obstructive sleep apnea, lung adenocarcinoma, vascular occlusion during severe bacterial infection.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property (ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

1. An isolated and purified nucleic acid comprising a nucleic acid encoding a mutant DNase 1L3 comprising at least one mutation for post-translational attachment of polyethylene-glycol (PEG) to the mutant DNase1L3 to increase the serum half-life of the mutant DNase1L3.

2. The nucleic acid of claim 1, further comprising at least one of:
a nucleic acid sequence optimized for microbial expression;
a nucleic acid segment encoding a leader sequence;
a nucleic acid segment encoding a leader sequence; or
a codon-optimized mutant DNase1L3 nucleic acid encoding SEQ ID NOS: 2-8, with the at least one: D38C, A126C, Y261C, N78C, S79C, R80C, K147C, or S245C mutation.

3. The nucleic acid of claim 1, wherein the mutant DNase1L3 comprises the mutant DNase1L3 comprises at least one of:
D38C, A126C, Y261C, N78C, S79C, R80C, K147C, or S245C mutation;
S91C, S112C, S131C, S253C, S272C, or S279C mutation; or
D38C, A126C, Y261C, N78C, S79C, R80C, K147C, or S245C mutation.

4. (canceled)

5. (canceled)

6. (canceled)

7. (canceled)

8. The nucleic acid of claim 1, wherein the nucleic acid encodes a protein that comprises an about 95, 96, 97, 98, 99, or 100 percent identity or higher with a codon-optimized mutant DNase1L3 of SEQ ID NO: 2-8, with the at least one: D38C, A126C, Y261C, N78C, S79C, R80C, K147C, or S245C mutation.

9. The nucleic acid of claim 1, further comprising an expression vector comprising a nucleic acid encoding the protein of SEQ ID NO: 2-8 for a mutant DNase 1L3 comprising at least one mutation for post-translational attachment of polyethylene-glycol (PEG) operably linked to a promoter recognized by a host cell transformed with the vector.

10. The nucleic acid of claim 9, wherein the host cell is a bacterial or yeast cell, or the host cell comprises E. coli or Pichia pastoris.

11. (canceled)

12. A mutant DNase1L3 having at least about a 95% identity with a nucleic acid sequence encoding a mutant DNase1L3 comprising at least one mutation for post-translational attachment of polyethylene-glycol (PEG) to the mutant DNase1L3 to increase the serum half-life of the mutant DNase1L3.

13. The mutant DNase1L3 of claim 12, further comprising at least one of:
a nucleic acid sequence optimized for microbial expression;
a nucleic acid segment encoding a leader sequence;
a nucleic acid segment encoding a leader sequence; or
a codon-optimized mutant DNase1L3 nucleic acid encoding SEQ ID NOS: 2-8, with the at least one: D38C, A126C, Y261C, N78C, S79C, R80C, K147C, or S245C mutation.

14. The mutant DNase1L3 of claim 12, wherein the mutant DNase1L3 comprises the mutant DNase1L3 comprises at least one of:
D38C, A126C, Y261C, N78C, S79C, R80C, K147C, or S245C mutation;
S91C, S112C, S131C, S253C, S272C, or S279C mutation; or
D38C, A126C, Y261C, N78C. S79C, R80C, K147C, or S245C mutation.

15. (canceled)

16. (canceled)

17. (canceled)

18. (canceled)

19. The mutant DNase1L3 of claim 12, wherein the nucleic acid encodes a mutant protein that comprises an about 95, 96, 97, 08, 99, or 100 percent identity or higher with a codon-optimized mutant DNase1L3 of SEQ ID NO: 2-8, with the at least one: D38C, A126C, Y261C, N78C, S79C, R80C, K147C, or S245C mutation.

20. The mutant DNase1L3 of claim 12, wherein the mutant DNase1L3 is post-translationally modified with polyethylene glycol, or the mutant DNase1L3 is post-translationally modified with a polyethylene glycol having a molecular mass from 5 kDa to 50 kDa.

21. (canceled)

22. A host cell transformed with an expression vector comprising a nucleic acid encoding an amino acid sequence of SEQ ID NO: 2-8 for a mutant DNase1L3, with the at least one: D38C, A126C, Y261C, N78C, S79C, R80C, K147C, or S245C mutation.

23. The host cell of claim 22, wherein the host cell comprises a bacterial or a yeast cell, wherein the host cell comprises E. coli, Pichia pastoris, or a host strain that allows enhanced disulfide bond formation and enhanced expression of eukaryotic proteins that contain codons rarely used in E. coli.

24. (canceled)

25. A method of making a protein with DNase activity comprising the steps of:
transforming a host cell with an isolated nucleic acid comprising a nucleotide sequence encoding a mutant DNase1L3 protein with at least about an 95% identity with SEQ ID NO: 2-8 for a DNase, with the at least one: D38C, A126C, Y261C, N78C, S79C, R80C, K147C, or S245C mutation; and
culturing the host cell under conditions such that the mutant DNase1L3 protein is produced by the host cell, wherein the mutant DNase1L3 protein comprises at least one mutation for post-translational modification or attachment of a molecule to the mutant DNase1L3 protein to increase the serum half-life of the mutant DNase1L3 protein.

26. The method of making of claim 25, wherein culturing a bacterial or yeast host cell transformed with an expression vector is further defined as comprising a DNA sequence comprising the nucleotide sequence encoding the mutant DNase1L3 of SEQ ID NO:2-8, with the at least one: D38C, A126C, Y261C, N78C, S79C, R80C, K147C, or S245C mutation, expressing the mutant DNase1L3 in the cultured yeast host cell; and
isolating the mutant DNase1L3.

27. The method of claim 26, wherein the mutant DNase1L3 is post-translationally modified with polyethylene glycol, or the mutant DNase1L3 is post-translationally modified with a polyethylene glycol having a molecular mass from 5 kDa to 50 kDa.

28. (canceled)

29. The method of claim 26, wherein the method further comprising the steps of:
transforming the host cell with a nucleic acid molecule that encodes the mutant DNase1L3 comprising an amino acid sequence of SEQ ID NO: 2-8, with the at least one: D38C, A126C, Y261C, N78C, S79C, R80C, K147C, or S245C mutation; and
culturing the host cell under conditions in which the mutant DNase1L3 is produced by the host cell.

30. The method of claim 29, wherein the host cell comprises E. coli or Pichia pastoris.

31. The method of claim 29, wherein the host cell produces at least 0.25 mg/L mutant DNase1L3 protein, or the host cell produces at least 7.5 mg/L mutant DNase1L3 protein in TB broth.

32. (canceled)

33. A mutant DNase1L3 made by the process of claim 25.

34. The mutant DNase1L3 of claim 33, wherein the mutant DNase1L3 is post-translationally modified with polyethylene glycol, or is post-translationally modified with a polyethylene glycol having a molecular mass from 5 kDa to 50 kDa.

35. (canceled)

36. A method of preventing or treating an autoimmune disease comprising:
identifying a subject in need of treatment for the autoimmune disease; and
providing an effective amount of a DNase I of SEQ ID NO: 2-8 sufficient to prevent or treat the autoimmune disease.
The method of claim 36, wherein the autoimmune disease is selected from at least one of: systemic lupus erythematosus, autoimmune liver disease, cystic fibrosis, autoimmune hepatitis, primary sclerosing cholangitis, primary biliary cirrhosis, rheumatoid arthritis, systemic sclerosis, scleroderma, asthma, dermatomyositis/polymyositis, autoimmune hemolytic anemia, hepatocellular carcinoma, ovarian cancer, hypocomplementemic urticarial vasculitis syndrome, Behcet's disease, COVID-19, ankylosing spondylitis, obstructive sleep apnea, lung adenocarcinoma, vascular occlusion during severe bacterial infection.
